(19) United States
(12) Patent Application Publication
(10) Pub. No.: US 2021/0252027 A1
(43) Pub. Date: Aug. 19, 2021

(54) PHARMACEUTICAL FORMULATION CONTAINING REMDESIVIR

(71) Applicants: Cai Gu Huang, Shrewsbury, MA (US); Ning He, Shanghai (CN)

(72) Inventors: Cai Gu Huang, Shrewsbury, MA (US); Ning He, Shanghai (CN)

(73) Assignee: Cai Gu Huang, Shrewsbury, MA (US)

(21) Appl. No.: 17/168,262

(22) Filed: Feb. 5, 2021

Related U.S. Application Data
(60) Provisional application No. 62/971,232, filed on Feb. 7, 2020.

(51) Int. Cl. A61K 31/675; A61K 47/40; A61K 47/18; A61K 9/00

(52) U.S. Cl. CPC A61K 31/675; A61K 47/40; A61K 9/0078; A61K 47/186; A61K 47/183

(57) ABSTRACT

The present invention relates to a liquid pharmaceutical preparation and a method for administering the pharmaceutical preparation by nebulization or soft mist inhalation. The pharmaceutical formulation comprises: (a) remdesivir or a pharmaceutically acceptable salt thereof; (b) an excipient selected from the group consisting of (i) a pharmacologically acceptable stabilizer or complexing agent and (ii) a solubility enhancing agent; and (c) a solvent wherein the pharmaceutical formulation has a pH of between about 2.0 and about 6.0.

### PRIORITY STATEMENT

This application claims the benefit of the filing date of U.S. Provisional Patent Application No. 62/971,232, filed on Feb. 7, 2020, which is incorporated herein by reference in its entirety.

### BACKGROUND OF THE INVENTION

Remdesivir, chemically 2-ethylbutyl((S)-(((2R,3S,4R,5R)-5-(4-aminopyrrolo[1,2-b]pyridazin-7-yl)-5-cyano-3,4-dihydroxytetrahydrofuran-2-yl) methoxy) (phenoxy) phosphoryl)-L-alaninate, has the following chemical structure:

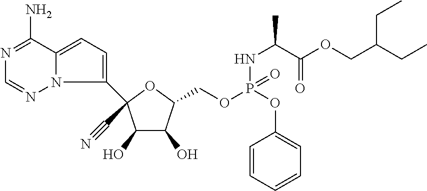 [SMILES: CCC(CC)COC(=O)[C@H](C)NP(=O)(OC[C@H]1O[C@@](C#N)(c2ccc3c(N)ncnn23)[C@H](O)[C@@H]1O)Oc1ccccc1]

Remdesivir has been found to show desirable antiviral activity against more distantly related viruses such as respiratory syncytial virus, Junin virus, Lassa fever virus, and severe acute respiratory syndrome associated coronavirus (SARS-CoV). It was rapidly pushed through clinical trials due to the West African Ebola virus epidemic crisis.

Remdesivir is active against a broad spectrum of viral pathogens, including Middle East Respiratory Syndrome (MERS) virus, SARS-CoV-2, Marburg virus, and multiple variants of Ebola virus, including the Makona strain causing the most recent outbreak in Western Africa. Recent studies showed remdesivir may be effective in treating the novel SARS-CoV-2 virus, which causes COVID-19.

Remdesivir is a nucleoside analog. After uptake into cells, it is converted to a nucleoside triphosphate which is incorporated by the virus's RNA-dependent RNA polymerase as the virus replicates. The nucleoside triphosphate to which remdesivir is converted is chemically different from adenosine triphosphate and blocks further nucleotides from being incorporated into the virus's growing RNA strand. RNA synthesis ceases, which blocks production of infectious virus particles.

Accordingly, remdesivir is a small-molecule antiviral agent that demonstrates robust therapeutic efficacy for preventing viral infection, and shows potential for broad-spectrum anti-virus activity.

SARS-CoV-2 mainly infects the respiratory tract, particularly causing respiratory illness and lung damage, and, in some cases, lung failure.

Remdesivir is currently formulated as a lyophilized dosage form for injection, now in clinical trials. One disadvantage of this formulation is that it results in little drug being delivered to the lungs. Another disadvantage is that the formulation must be infused over an extended period of time, which is inconvenient and results in adverse side effects.

### SUMMARY OF THE INVENTION

A novel, surprising approach to a more effective and selective method of delivering remdesivir to the lungs has been found, which more effectively inhibits and removes the virus from the lungs and other parts of the human body. The novel remdesivir delivery method, comprising soft mist inhalation or nebulization inhalation, presents clear and significant clinical benefits, including higher efficacy and fewer adverse side effects.

The present invention relates to pharmaceutical formulations of remdesivir and pharmaceutically acceptable salts, solvates, or metabolites thereof, which can be administered by soft mist or nebulization inhalation. The pharmaceutical formulations of the invention have to meet high quality standards.

One aspect of the present invention comprises providing a pharmaceutical formulation containing remdesivir and inactive ingredients, which meets high quality standards and is able to achieve optimum nebulization of a solution using a soft mist inhaler. It is desirable that the active ingredient in the formulation be pharmaceutically stable in storage for a time period of a few months or years, such as 1-6 months, one year, or three years, at a temperature of from about 2° C. to about 8° C.

Another aspect of the present invention is providing formulations of solutions containing remdesivir, which are nebulized under pressure using an inhaler, such as a soft mist inhaler device. In an embodiment, the formulation delivered by the inhaler is an aerosol having particle sizes that reproducibly fall within a specified range.

Another aspect of the present invention is providing nebulization formulations comprising remdesivir and inactive excipients, which can be administered by nebulization inhalation using ultrasonic-based, air pressure-based, or mesh-based nebulizers/inhalers. It is desirable that the active ingredient or ingredients in the formulation be pharmaceutically stable in storage for a time period of a few months or years, such as 1-6 months, one year, or three years, at a temperature of from about 2° C. to about 8° C.

These and other features, aspects and advantages of the present invention will become better understood with reference to the following description and appended claims. The accompanying drawings, which are incorporated in and constitute a part of this specification, illustrate embodiments of the invention and, together with the description, serve to explain the principles of the invention.

### BRIEF DESCRIPTION OF THE DRAWINGS

FIG. 1 is a longitudinal section through the atomizer in the stressed state.

FIG. 2 is a counter element of the atomizer.

FIG. 3 is a graph of the particle size distribution of remdesivir as described in example 3.

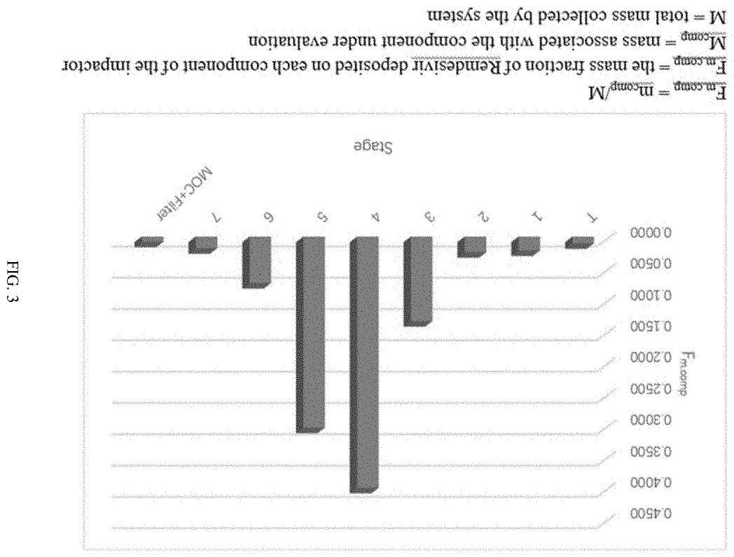

FIG. 4 is a graph of particle size distribution of droplets sprayed by a mesh-based atomizer as described in example 4.

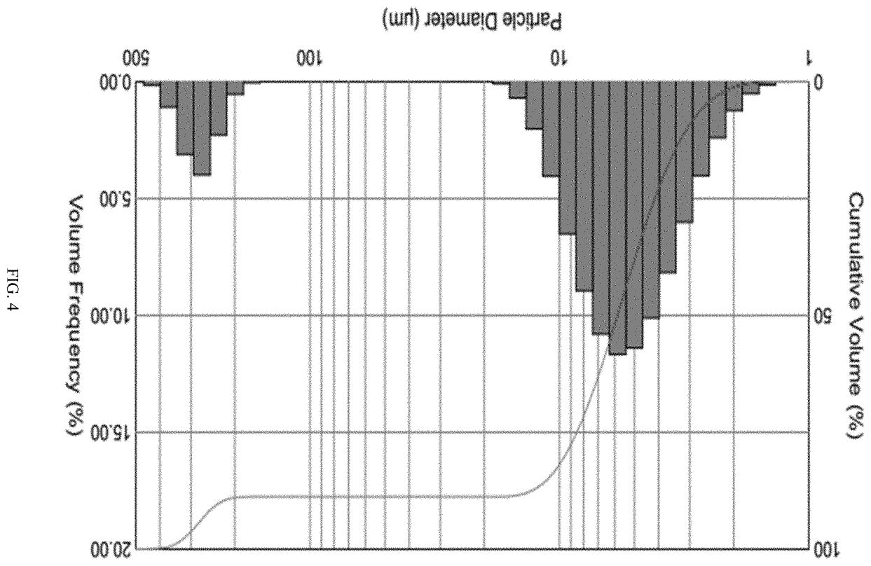

FIG. 5 is an illustrative chromatogram depicting the retention time of impurity 1 as described in example 1.

### DETAILED DESCRIPTION OF THE INVENTION

For purposes of describing the invention, reference now will be made in detail to embodiments and/or methods of the invention, one or more examples of which are illustrated in or with the drawings. Each example is provided by way of explanation of the invention, not limitation of the invention. In fact, it will be apparent to those skilled in the art that various modifications and variations can be made in the present invention without departing from the scope or spirit of the invention. For instance, features or steps illustrated or described as part of one embodiment, can be used with another embodiment or steps to yield a still further embodiments or methods. Thus, it is intended that the present invention covers such modifications and variations as come within the scope of the appended claims and their equivalents.

It is advantageous to achieve better delivery of active substance(s) to the lungs for the treatment of lung diseases. Furthermore, it is important to maximize deposition of a drug in the lungs when the drug is delivered by inhalation.

Therefore, there is a need to increase lung deposition when administering a drug by inhalation delivery. The soft mist or nebulization inhalation device disclosed in US2019/0030268 can significantly increase the lung deposition of inhalable drugs.

The inhalers disclosed in US2019/0030268 nebulize a small amount of a liquid formulation within a few seconds into an aerosol that is suitable for therapeutic inhalation. The inhalers are particularly suitable for the liquid formulations disclosed herein.

In an embodiment, soft mist or nebulization devices for administering the pharmaceutical formulations of the present invention are those in which an amount of less than about 70 microliters of pharmaceutical solution can be nebulized in one puff, such as less than about 30 microliters, for example less than about 15 microliters, so a therapeutically effective quantity of the aerosol is inhaled. An average particle size of the aerosol formed from one puff is less than about 15 microns, such as less than about 10 microns.

In an embodiment, the nebulization devices for administering the pharmaceutical formulations of the present invention are those in which an amount of less than about 8 milliliters of pharmaceutical solution can be nebulized in one puff, such as less than about 2 milliliters, for example less than about 1 milliliter, so a therapeutically effective quantity of the aerosol is inhaled. An average particle size of the aerosol formed from one puff is less than about 15 microns, such as less than about 10 microns.

A device of this kind for the propellant-free administration of a metered amount of a liquid pharmaceutical composition for inhalation is described in detail, for example, in US2019/0030268 “Inhalation Atomizer Comprising a Blocking Function and a Counter”.

The pharmaceutical formulation in the nebulizer is converted into an aerosol destined for the lungs. The pharmaceutical formulation is sprayed by the nebulizer using high pressure.

The pharmaceutical formulations are stored in a reservoir in these inhalers. In an embodiment, the pharmaceutical formulations do not contain any ingredients which might interact with the inhaler to affect the pharmaceutical quality of the formulation or of the aerosol produced. In an embodiment, the active substance(s) in the pharmaceutical formulations are stable when stored and can be administered directly.

In one embodiment, the pharmaceutical formulations of the invention, which may be administered using the inhaler described above, contain additives, such as the disodium salt of edetic acid (sodium edetate), to reduce the incidence of spray anomalies and to stabilize the formulations. In one embodiment, the pharmaceutical formulations have a minimum concentration of sodium edetate.

One aspect of the present invention is to provide a pharmaceutical formulation containing remdesivir and excipients, which meets the high standards needed to achieve optimum nebulization of a solution using a soft mist inhaler. It is desirable that the active ingredient or ingredients in the formulation be pharmaceutically stable in storage for a time period of a few months or years, such as 1-6 months, one year, or three years.

Another aspect of the invention is to provide pharmaceutical formulations containing remdesivir, which are nebulized under pressure using an inhaler, such as a soft mist inhaler device. In an embodiment, the formulation delivered by the inhaler is an aerosol having particle sizes that reproducibly fall within a specified range.

Another aspect of the invention is to provide a pharmaceutical formulation comprising remdesivir and inactive excipients which can be administered by nebulization inhalation. In one embodiment the active ingredient of remdesivir comprises particles having a mass median aerodynamic diameter of about 1 micron to about 5 microns. These size particles are able to penetrate the lung upon inhalation.

In an embodiment, the pharmaceutical formulations of the invention comprise an active substance selected from the group consisting of remdesivir, its pharmaceutically acceptable salts, its pharmaceutically acceptable solvates, and its pharmaceutically acceptable active metabolites.

In an embodiment, the active substance is dissolved in a solvent. In one embodiment, the solvent may be water.

The current invention provides a method of treating viral infection in a patient, wherein the viral infection is selected from Ebola and Marburg virus (Filoviridae), coronavirus, SARS-CoV-2, Ross River virus, chikungunya virus, Sindbis virus, eastern equine encephalitis virus (Togaviridae, Alphavirus), vesicular stomatitis virus (Rhabdoviridae, Vesiculovirus), Amapari virus, Pichindé virus, Tacaribe virus, Junin virus, Machupo virus (Arenaviridae, Mammarenavirus), West Nile virus, dengue virus, yellow fever virus (Flaviviridae, Flavivirus), human immunodeficiency virus type 1 (Retroviridae, Lentivirus), Moloney murine leukemia virus (Retroviridae, Gammaretrovirus), influenza A virus (Orthomyxoviridae), respiratory syncytial virus (Paramyxoviridae, Pneumovirinae, Pneumovirus), vaccinia virus (Poxviridae, Chordopoxvirinae, Orthopoxvirus), herpes simplex virus type 1, herpes simplex virus type 2 (Herpesviridae, Alphaherpesvirinae, Simplexvirus), human cytomegalovirus (Herpesviridae, Betaherpesvirinae, Cytomegalovirus), Autographa californica nucleopolyhedrovirus (Baculoviridae, Alphabaculoviridae) (an insect virus), Semliki Forest virus, O'nyong-nyong virus, Sindbis virus, eastern/western/Venezuelan equine encephalitis virus (Togaviridae, Alphavirus), rubella (German measles) virus (Togaviridae, Rubivirus), rabies virus, Lagos bat virus, Mokola virus (Rhabdoviridae, Lyssavirus), Guanarito virus, Sabia virus, Lassa virus (Arenaviridae, Mammarenavirus), Zika virus, Japanese encephalitis virus, St. Louis encephalitis virus, tick-borne encephalitis virus, Omsk hemorrhagic fever virus, Kyasanur Forest virus (Flaviviridae, Flavivirus), human hepatitis C virus (Flaviviridae, Hepacivirus), influenza AB virus (Orthomyxoviridae, the common ‘flu’ virus), respiratory syncytial virus (Paramyxoviridae, Pneumovirinae, Pneumovirus), Hendra virus, Nipah virus (Paramyxoviridae, Paramyxovirinae, Henipavirus), measles virus (Paramyxoviridae, Paramyxovirinae, Morbillivirus), variola major (smallpox) virus (Poxviridae, Chordopoxvirinae, Orthopoxvirus), human hepatitis B virus (Hepadnaviridae, Orthohepadnavirus), hepatitis delta virus (hepatitis D virus), Middle East Respiratory Syndrome (MERS) virus, severe acute respiratory syndrome CoV (SARS-CoV).

The effective dose of remdesivir or its pharmaceutically acceptable salts or its active metabolites against SARS-CoV-2 depends on its bioavailability and clinical efficacy. The effective dose of remdesivir or its pharmaceutically acceptable salts against SARS-CoV-2 is between about 1 mg and about 100 mg, such as between about 10 mg and about 50 mg, for example between about 20 mg and about 30 mg.

The concentration of the remdesivir or its pharmaceutically acceptable salts in the finished pharmaceutical formulation depends on the desired therapeutic effects.

In an embodiment, the concentration of remdesivir or its pharmaceutically acceptable salts in a pharmaceutical formulation for administration by soft mist inhalation is between about 1 g/100 ml and about 20 g/100 ml, such as between about 10 g/100 ml and about 15 g/100 ml. In one embodiment, the soft mist devices for administration of the pharmaceutical formulation of the present invention can atomize a pharmaceutical solution of about 10 microliters to about 15 microliters, which may be administered so a therapeutically effective quantity of the aerosol is inhaled.

In one embodiment, the pharmaceutical formulation further comprises a pH adjusting agent, such as an acid or a base. The pH adjusting agent may be selected from pharmaceutically acceptable acids and bases. In one embodiment, the pH adjusting agent is selected from the group consisting of hydrochloric acid and sodium hydroxide.

In another embodiment, the pH adjusting agents is selected from the group consisting of citric acid and sodium hydroxide.

Maintaining the pH within a desired range helps to maintain the stability of the active substance(s), the excipients, or both the active substance(s) and excipients. In an embodiment, the pH of the pharmaceutical formulations of the current invention is in the range of about 2.0 to about 6.0. In one embodiment, the pH is in the range of about 3.0 to about 5.0. In another embodiment, the pH is in the range of about 3.5 to about 4.5.

In an embodiment, the pharmaceutical formulation further comprises a stabilizer or complexing agent. In one embodiment, the stabilizer or complexing agent is edetic acid (EDTA) or one of the known salts thereof, disodium edetate or edetate disodium dihydrate. In an embodiment, the pharmaceutical formulation contains one or more of edetic acid and salts thereof.

The pharmaceutical formulation may contain pharmaceutically acceptable stabilizers or complexing agents suitable for use in formulations for inhalation. In one embodiment, the stabilizers or complexing agents are selected from the group consisting of citric acid, edetate disodium, edetate disodium dihydrate, and combinations thereof.

A complexing agent is a molecule capable of entering into complex bonds. In an embodiment, these compounds have the effect of complexing cations. In one embodiment, the concentration of the stabilizers or complexing agents is about 1 mg/100 ml to about 500 mg/100 ml. In another embodiment, the concentration of the stabilizers or complexing agents is about 5 mg/100 ml to about 200 mg/100 ml. In another embodiment, the complexing agent is edetate disodium dihydrate at a concentration of about 10 mg/100 ml.

In an embodiment, all the ingredients of the pharmaceutical formulation are present in solution.

The pharmaceutical formulations may further comprise additives. The term “additives” as used herein means any pharmacologically acceptable and therapeutically useful substance, which is not an active substance, but can be formulated together with the active substances in a suitable solvent, in order to improve the qualities of the pharmaceutical formulation. In an embodiment, these substances have no appreciable pharmacological effects or undesirable pharmacological effects in the context of the desired therapy.

The additives include, for example, but are not limited to, other stabilizers, complexing agents, antioxidants, surfactants, preservatives which prolong the shelf life of the finished pharmaceutical formulation, vitamins and/or other additives known in the art.

In one aspect of the invention, the formulations further comprise at least one suitable preservative to protect the formulation from contamination with pathogenic bacteria. In an embodiment, the preservative comprises benzalkonium chloride, benzoic acid, sodium benzoate, or combinations thereof. In one embodiment, the only preservative in the formulation is benzalkonium chloride. The at least one preservative is typically present in the formulation in an amount of about 2 mg/100 ml to about 300 mg/100 ml. In one embodiment, the content of benzalkonium chloride is about 10 mg/100 ml.

In one aspect of the invention, the formulations further comprise at least one solubility enhancing agent to aid the solubility of the active ingredient or other excipients. In an embodiment, solubility enhancing agents include, but are not limited to, cyclodextrin derivatives, such as sulfobutylether β-cyclodextrin; or one of the known pharmaceutically acceptable salts thereof. In an embodiment, the formulation comprises sulfobutylether β-cyclodextrin and/or the salts thereof.

In another embodiment, the pharmaceutical formulation comprising a solubility enhancing agent is suitable for administration by soft mist inhalation. In an embodiment, the solubility enhancing agent includes, but is not limited to, polysorbate 20; polysorbate 80; poloxamer; cyclodextrin derivatives; sodium dodecyl sulfate (SDS); sodium laurel sulfate; sodium octyl glycoside; polyethyl glycol; polypropyl glycol; copolymers; and combinations thereof. In one embodiment, the solubility enhancing agent is one or more cyclodextrin derivatives or one of the known salts thereof. In an embodiment, the solubility enhancing agent is sulfobutylether β-cyclodextrin or the known pharmaceutically acceptable salts thereof.

Another aspect of the current invention is to provide stable pharmaceutical formulations containing remdesivir and excipients which can be administered by soft mist inhalation using atomizer inhalers. In an embodiment, the pharmaceutical formulation is stable for a time period of a few months or years, such as about 1 month to about 6 months, about one year, or about three years, and at a temperature of from about 2° C. to about 8° C. In one embodiment, the pharmaceutical formulation is stable for a time period of three years at a temperature of from about 2° C. to about 8° C.

Another aspect of the current invention is to provide pharmaceutical formulations comprising remdesivir and excipients which can be administered by nebulization inhalation using ultrasonic-based, air pressure-based, mesh-based nebulizers/inhalers. In an embodiment, the pharmaceutical formulation is stable for a time period of a few months or years, such as about 1 month to about 6 months, about one year, or about three years, and at a temperature below about 10° C. In one embodiment, the pharmaceutical formulation is stable for a time period of three years at a temperature of from about 2° C. to about 8° C.

Another aspect of the current invention is to provide nebulization formulations comprising sodium chloride. In an embodiment, the concentration of sodium chloride is about 0.1 g/100 ml to about 0.9 g/100 ml.

In an embodiment, the active ingredient concentration of remdesivir in the pharmaceutical formulation is between about 100 mg/100 ml and about 10 g/100 ml, such as between about 1,000 mg/100 ml and about 5,000 mg/100 ml.

In another aspect of the invention, the pharmaceutical formulations according to the invention comprise a solubility enhancing agent to aid the solubility of the active ingredient or other excipients. In one embodiment, the pharmaceutical formulation comprising a solubility enhancing agent is suitable for administration by nebulization inhalation. In an embodiment, the solubility enhancing agent includes, but is not limited to, polysorbate 20; polysorbate 80; poloxamer; cyclodextrin derivatives; sodium dodecyl sulfate (SDS); sodium laurel sulfate; sodium octyl glycoside; polyethyl glycol; polypropyl glycol; copolymers; and any mixture thereof. In one embodiment, the solubility enhancing agent is one or more cyclodextrin derivatives or one of the known salts thereof. In an embodiment, the solubility enhancing agent is sulfobutylether β-cyclodextrin and/or the salts thereof.

In an embodiment, the pharmaceutical formulation is stable for a storage time of a few months or years, such as about 1 month to about 6 months, about one year, or about three years, and at a temperature below about 10° C. In one embodiment, the pharmaceutical formulation is stable for a time period of about three years at a temperature of from about 2° C. to about 8° C.

Maintaining the pH within a desired range helps to achieve stability for the pharmaceutical formulation and to maintain the solubility of remdesivir. In one embodiment, the pH can be adjusted to the desired pH by adding an acid, including but not limited to, HCl, or by adding a base, including but not limited to, NaOH or by adding a combination of acid(s) and base(s), including but not limited to HCl and NaOH, to achieve the desired buffer concentration and pH value.

In one aspect of the invention, the pharmaceutical formulation has a pH value in the range of from about 3 to about 5. In one embodiment, the pharmaceutical formulation having a pH value in the range of from about 3 to about 5 is suitable for administration by nebulization inhalation. In another embodiment, the pharmaceutical formulation having a pH value in the range of from about 3 to about 5 is suitable for administration by soft mist inhalation.

In an embodiment, the pharmaceutical formulations can be filled into canisters for use in nebulization devices. In an embodiment, the pharmaceutical formulations exhibit substantially no particle growth or change of morphology. In an embodiment, there is no, or substantially no, deposition of suspended particles on the surfaces of either the canisters or the valves, and so the formulations can be discharged from a suitable nebulization device and deliver doses with a high degree of uniformity. In an embodiment, the suitable nebulization device is an ultrasonic vibrating mesh nebulizer, a compressed air nebulizer such as the Pari eFlow nebulization inhaler, or another commercially available ultrasonic nebulizer, jet nebulizer or mesh nebulizer.

In another aspect of the present invention, the pharmaceutical formulation is suitable for administration using a soft mist inhaler. In one embodiment, the pharmaceutical formulation containing remdesivir is used with an inhaler of the kind described herein.

The inhaler disclosed in US2019/0030268 is an example of an inhaler that is suitable for use with the formulations of the present invention.

The pharmaceutical formulation in the nebulizer is converted into an aerosol destined for the lungs. The nebulizer uses high pressure to spray the pharmaceutical formulation.

The soft mist inhalable device can be carried anywhere by the patient, since it has a cylindrical shape and a convenient size of less than about 8 cm to about 18 cm long, and about 2.5 cm to about 5 cm wide. The nebulizer sprays a defined volume of the pharmaceutical formulation out through small nozzles at high pressure, so as to produce an inhalable aerosol.

In an embodiment, the inhalation device comprises an atomizer 1, a fluid 2, a vessel 3, a fluid compartment 4, a pressure generator 5, a holder 6, a drive spring 7, a delivering tube 9, a non-return valve 10, a pressure room 11, a nozzle 12, a mouthpiece 13, an aerosol 14, an air inlet 15, an upper shell 16, and an inside part 17.

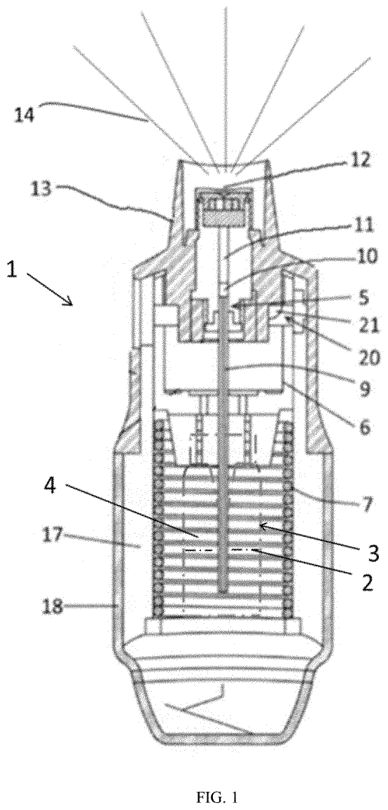

The inhalation atomizer 1 comprising the block function and the counter described above for spraying a medicament fluid 2 is shown in FIG. 1 in the stressed state. The atomizer 1 comprising the block function and the counter is a portable inhaler and requires no propellant gas.

FIG. 1 shows a longitudinal section through the atomizer in the stressed state.

For a typical atomizer 1 described above, an aerosol 14 that can be inhaled by a patient is generated through atomization of the fluid 2, which is preferably formulated as a medicament liquid. In an embodiment, the medicament is administered at least once a day. In another embodiment, the medicament is administered multiple times a day, preferably at predetermined time intervals, according to how seriously the illness affects the patient.

In an embodiment, the atomizer 1 described above has a substitutable and insertable vessel 3, which contains the medicament fluid 2. Therefore, a reservoir for holding the fluid 2 is formed in the vessel 3. Specifically, the medicament fluid 2 is located in the fluid compartment 4 formed by a collapsible bag in the vessel 3.

In an embodiment, the amount of fluid 2 for the inhalation atomizer 1 described above is in the vessel 3 to provide, for example, up to about 200 doses. A classical vessel 3 has a volume of about 2 ml to about 10 ml. A pressure generator 5 in the atomizer 1 is used to deliver and atomize the fluid 2, preferably in a predetermined dosage amount. Therefore, the fluid 2 can be released and sprayed in individual doses, such as from about 5 microliters to about 30 microliters.

In an embodiment, the atomizer 1 described above has a pressure generator 5, a holder 6, a drive spring 7, a delivering tube 9, a non-return valve 10, a pressure room 11, and a nozzle 12 in the area of a mouthpiece 13. The vessel 3 is latched by the holder 6 in the atomizer 1 so that the delivering tube 9 is plunged into the vessel 3. The vessel 3 can be separated from the atomizer 1 for substitution.

In an embodiment, when drive spring 7 is stressed in an axial direction, the delivering tube 9, the vessel 3, and the holder 6 will be shifted downwards. Then the fluid 2 will be sucked into the pressure room 11 through the delivering tube 9 and the non-return valve 10.

In an embodiment, after releasing the holder 6, the stress is eased. During this process, the delivering tube 9 and closed non-return valve 10 are shifted upward by releasing the drive spring 7. Consequently, the fluid 2 is under the pressure in the pressure room 11. The fluid 2 is then pushed through the nozzle 12 and atomized into an aerosol 14 by the resulting pressure. A patient can inhale the aerosol 14 through the mouthpiece 13, while the air is sucked into the mouthpiece 13 through air inlet 15.

In an embodiment, the atomizer 1 described above has an upper shell 16 and an inside part 17, which can be rotated relative to the upper shell 16. A lower shell 18 is manually operable to attach to the inside part 17. The lower shell 18 can be separated from the atomizer 1 so that the vessel 3 can be substituted and inserted.

In an embodiment, the atomizer 1 described above has a lower shell 18, which carries the inside part 17, and is rotatable relative to the upper shell 16. As a result of rotation and engagement between the upper unit 17 and the holder 6, through a gear 20, the holder 6 axially moves the counter in response to the force of the drive spring 7, and the drive spring 7 is stressed.

In an embodiment, in the stressed state, the vessel 3 is shifted downwards until it reaches a final position, which is shown in FIG. 1. The drive spring 7 is stressed in this final position. The holder 6 is then clasped. Therefore, the vessel 3 and the delivering tube 9 are prevented from moving upwards so that the drive spring 7 is stopped from easing.

In an embodiment, the atomizing process occurs after the holder 6 is released. The vessel 3, the delivering tube 9, and the holder 6 are shifted by the drive spring 7 to the beginning position. This is referred to herein as major shifting. While the major shifting occurs, the non-return valve 10 is closed and the fluid 2 is under pressure in the pressure room 11 by the delivering tube 9, and the fluid 2 is then pushed out and atomized by the pressure.

In an embodiment, the atomizer 1 described above may have a clamping function. During the clamping, the vessel 3 preferably performs a lifting shift for the withdrawal of the fluid 2 during the atomizing process. The gear 20 has sliding surfaces 21 on the upper shell 16 and/or on the holder 6, which allows holder 6 to move axially when the holder 6 is rotated relative to the upper shell 16.

In an embodiment, the holder 6 is not blocked for too long and can carry on the major shifting. Therefore, the fluid 2 is pushed out and atomized.

In an embodiment, when the holder 6 is in the clamping position, the sliding surfaces 21 move out of engagement. Then the gear 20 releases the holder 6 for the opposite axial shift.

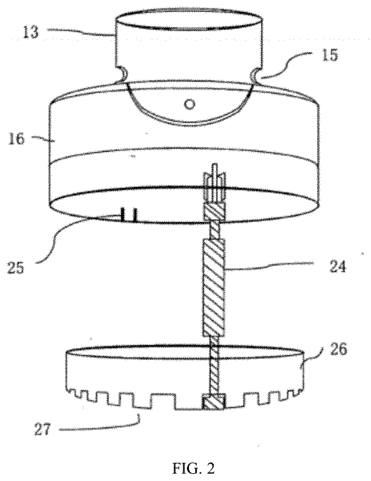

In an embodiment, the atomizer 1 includes a counter element shown in FIG. 2. The counter element has a worm 24 and a counter ring 26. In an embodiment, the counter ring 26 is circular and has a dentate part at the bottom. The worm 24 has upper and lower end gears. The upper end gear contacts with the upper shell 16. The upper shell 16 has inside bulge 25. When the atomizer 1 is employed, the upper shell 16 rotates; and when the bulge 25 passes through the upper end gear of the worm 24, the worm 24 is driven to rotate. The rotation of the worm 24 drives the rotation of the counter ring 26 through the lower end gear so as to result in a counting effect.

In an embodiment, the locking mechanism is realized mainly by two protrusions. Protrusion A is located on the outer wall of the lower unit of the inside part. Protrusion B is located on the inner wall of counter. The lower unit of the inside part is nested in the counter. In on embodiment, the counter rotates relative to the lower unit of the inside part. Because of the rotation of the counter, the number displayed on the counter changes as the actuation number increases, which can be observed by the patient. After each actuation, the number displayed on the counter changes. Once the predetermined number of actuations is achieved, Protrusion A and Protrusion B will encounter each other and the counter will be prevented from further rotation. This blocks the atomizer, stopping it from further use. The number of actuations of the device can be counted by the counter.

The nebulizer described above is suitable for nebulizing the pharmaceutical formulations according to the invention to form an aerosol suitable for inhalation. Nevertheless, the formulations according to the invention can also be nebulized using other inhalers apart from those described above, such as an ultrasonic vibrating mesh nebulizer and a compressed air nebulizer.

Atomization devices include, but are not limited, to soft mist inhalers, ultrasonic atomizers, air compression atomizers, and mesh-based atomizers.

In an embodiment of the invention, a soft mist inhaler is used. The soft mist inhaler provides pressure to eject a metered dose of drug solution. In one embodiment, two high-speed jets are formed, and the two jets collide with each other to form droplets with smaller particles.

In another embodiment of the invention, an ultrasonic atomizer is used. The oscillation signal of the main circuit board of an ultrasonic atomizer is amplified by a high-power triode and transmitted to an ultrasonic wafer. The ultrasonic wafer converts electrical energy into ultrasonic energy. The ultrasonic energy atomizes the water-soluble drug into tiny mist particles of about 1 um to about 5 um at normal temperature. With the help of an internal fan, the particles are ejected.

In another embodiment of the invention, an air compression atomizer is used. The air compression atomizer comprises a compressed air source and an atomizer. The compressed air is decompressed after passing through a narrow opening at high speed, a negative pressure is generated locally, and the drug solution is sucked out from the container because of the siphon effect. At a high-speed air flow, the air is broken into small aerosol particles by collision.

In another embodiment of the invention, a mesh based atomizer is used. A mesh based atomizer contains a stainless-steel mesh covered with micropores having a diameter of about 3 μm. The number of micropores exceeds about 1,000. The mesh is conical in shape, with the cone bottom facing the liquid surface. Under pressure, the vibration frequency of the mesh is about 130 KHz. High vibration frequency breaks the surface tension of the drug solution contacted with the mesh and produces a low-speed aerosol.

### EXAMPLES

Materials and Reagents:

50% benzalkonium chloride aqueous solution purchased from Merck
Edetate disodium dihydrate purchased from Merck
Sodium hydroxide purchased from Titan reagents
Hydrochloric acid purchased from Titan reagents
Sulfobutylether β-Cyclodextrin (referred to as SBECD) purchased from Zhiyuan Bio-tech Co., Ltd., China
Remdesivir obtained from Nanchang Anovent Pharmaceutical Co., Ltd.

### Example 1

The formulation and preparation of the nebulization inhalation solutions (samples 1-5) are as follows:

SBECD according to the amount provided in table 1 was dissolved in 35 ml purified water. The solution was adjusted to pH 1.9 with hydrochloric acid. Remdesivir (referred to as RV) according to the amount provided in table 1 was added to the solution, then the solution was adjusted to pH 2.0 with hydrochloric acid and sonicated until completely dissolved. The solution was adjusted to the target pH shown in table 1 with hydrochloric acid or sodium hydroxide. Finally, purified water was added to produce a final volume of 40 ml.

TABLE 1
Ingredient contents of samples 1-5

| Ingredients | Sample 1 | | Sample 2 | | Sample 3 | | Sample 4 | | Sample 5 | |
|---|---|---|---|---|---|---|---|---|---|---|
| Remdesivir (RV) | 200 | mg | 200 | mg | 200 | mg | 200 | mg | 200 | mg |
| Sulfobutyl ether β- | 4.0 | g | 4.0 | g | 4.0 | g | 4.0 | g | 4.0 | g |
| Cyclodextrin (SBECD) | | | | | | | | | | |

| | | | | | |
|---|---|---|---|---|---|
| Hydrochloric acid or | To pH 3.0 | To pH 3.5 | To pH 4.0 | To pH 4.5 | To pH 5.0 |
| sodium hydroxide | | | | | |
| Purified water | Added to | Added to | Added to | Added to | Added to |
| | 40 ml | 40 ml | 40 ml | 40 ml | 40 ml |

TABLE 2
Stability at different pH values(conditions: 40° C. ± 2° C./75% ± 5% RH)

| | | | Sample 1 | Sample 2 | Sample 3 | Sample 4 | Sample 5 |
|---|---|---|---|---|---|---|---|
| Formulation | | | pH 3.0 | pH 3.5 | pH 4.0 | pH 4.5 | pH 5.0 |
| 0 day | characteristics | | Colorless | Colorless | Colorless | Colorless | Colorless |
| | | | clear | clear | clear | clear | clear |
| | | | solution | solution | solution | solution | solution |
| | RV | concentration | 5.011 | 4.941 | 4.947 | 4.819 | 4.891 |
| | | mg/ml | | | | | |
| | | recovery % | 100.22 | 98.71 | 98.94 | 96.37 | 97.81 |
| | Impurity % | Impurity 1 | 0.348 | 0.217 | 0.145 | 0.104 | 0.264 |
| | | (RRT = 0.604) | | | | | |
| | | unknown | 0.348 | 0.217 | 0.145 | 0.104 | 0.264 |
| | | maximum | | | | | |
| | | impurity | | | | | |
| | | Total | 0.432 | 0.292 | 0.199 | 0.165 | 0.376 |
| | | impurities | | | | | |
| 40° C. | characteristics | | Colorless | Colorless | RV | RV | RV |
| 5 days | | | clear | clear | precipitation | precipitation | precipitation |
| | | | solution | solution | | | |
| | RV | concentration | 4.759 | 4.829 | 3.172 | 1.822 | 1.109 |
| | | mg/ml | | | | | |
| | | recovery % | 95.19 | 96.59 | 63.45 | 36.45 | 22.19 |
| | Impurity % | Impurity 1 | 2.311 | 1.048 | 0. 807 | 0.616 | 0.47 |
| | | (RRT = 0.604) | | | | | |
| | | unknown maximum | 2.311 | 1.048 | 0.807 | 0.616 | 0.47 |
| | | impurity | | | | | |
| | | Total impurities | 2.764 | 1.391 | 1.125 | 0.886 | 0.904 |
| 40° C. | Characteristics | | Colorless | Colorless | RV | RV | RV |
| 10 days | | | clear | clear | precipitation | precipitation | precipitation |
| | | | solution | solution | | | |
| | RV | concentration | 4.567 | 4.821 | RV | RV | RV |
| | | mg/ml | | | precipitation | precipitation | precipitation |
| | | recovery % | 91.31 | 96.41 | | | |
| | Impurity % | impurity1 | 4.896 | 2.103 | | | |
| | | (RRT = 0.604) | | | | | |
| | | unknown maximum | 4.896 | 2.103 | | | |
| | | impurity | | | | | |
| | | Total impurities | 5.677 | 2.596 | | | |

Analysis method for impurity 1:

Chromatographic column: YMC Triart C18, 5 μm, 4.6*150 mm
Flow: 1.0 ml/min
Column temperature: 35° C.

### Wavelength: 240 nm

Sample volume: 10 μl

### Time: 35 min

Gradient elution:

| Time (min) | A % | B % |
|---|---|---|
| 0 | 95 | 5 |
| 5 | 95 | 5 |
| 15 | 55 | 45 |
| 25 | 10 | 90 |
| 28 | 10 | 90 |
| 28.2 | 95 | 5 |
| 35 | 95 | 5 |

Mobile phase:
Mobile phase A: 1 L water+0.1% H₃PO₄
Mobile phase B: ACN

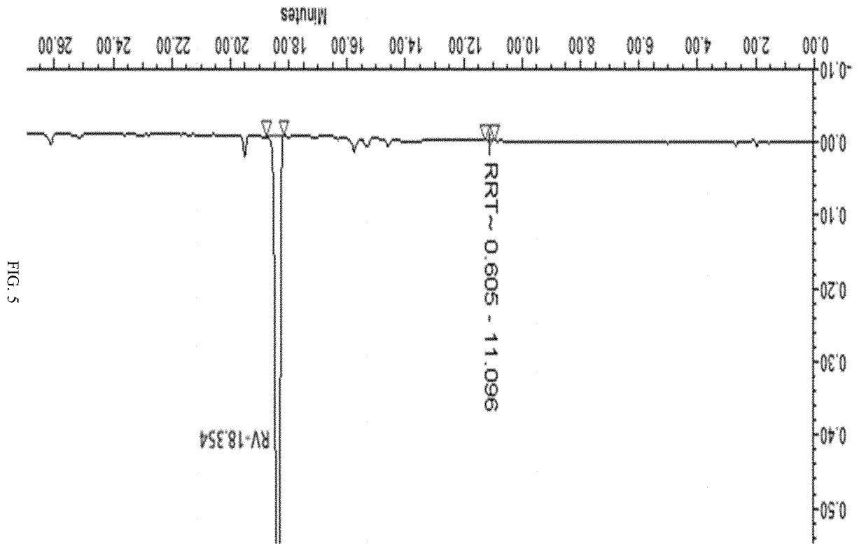

An illustrative chromatogram for impurity 1 is shown in FIG. 5. The relative retention time of impurity 1 is 0.605.

The above studies confirmed that the stability of a remdesivir solution is highly dependent on the formulation pH. As can be seen from Table 2, the remdesivir formulations are stable at pH 3-5, with the highest stability at pH 3.5-4.5.

### Example 2

Solubility of Remdesivir in Water Containing SBECD:

The solubility of remdesivir in water is extremely low. SBECD is added as a cosolvent. Remdesivir has good solubility in acidic conditions, exhibiting better solubility below pH 2. The results are shown in Table 3.

TABLE 3
Remdesivir solubility in different pH and SBECD

| | pH | | | | | | | | | |
|---|---|---|---|---|---|---|---|---|---|---|
| SBECD | 3 | | 3.5 | | 4 | | 4.5 | | 5 | |
| 5% SBECD | ≤10 | mg/ml | <7 | mg/ml | <6 | mg/ml | <5 | mg/ml | ≤2.5 | mg/ml |
| 10% SBECD | >20 | mg/ml | ≤20 | mg/ml | ≤10 | mg/ml | 5 | mg/ml | ≤5 | mg/ml |

ND: not detected

Preparation of 5% SBECD: 5 g of SBECD was dissolved in 100 ml remdesivir solution.

Preparation of 10% SBECD: 10 g of SBECD was dissolved in 100 ml remdesivir solution.

The above studies confirmed that the solubility of remdesivir is highly dependent on the formulation pH and the concentration of SBECD. Remdesivir exhibited better solubility with SBECD at a concentration of 10% compared to 5%.

TABLE 4
Solubility of remdesivir in tween-80:

| Solvent | RV concentration (mg/ml) |
|---|---|
| pH = 3.0 (0.2 mg/ml tween-80) | 0.0594 |
| pH = 3.0 (5% tween-80) | 1.1911 |
| pH = 4.0 (0.2 mg/ml tween-80) | 0.0221 |
| pH = 4.0 (5% tween-80) | 0.9934 |
| pH = 5.0 (0.2 mg/ml tween-80) | 0.0275 |
| pH = 5.0 (5% tween-80) | 0.9743 |

Preparation of 5% tween-80: 5 g of tween-80 were dissolved in 100 ml remdesivir solution.

Results and discussion: Tween-80 as a co-solvent does not meet the dosage requirements.

### Example 3

The formulation and preparation of the nebulization inhalation solutions (sample 6-7) are as follows:

Sodium chloride and SBECD according to the amounts provided in table 5, were dissolved in 90 ml purified water, The solution was adjusted to pH 1.9 with hydrochloric acid. Remdesivir according to the amount provided in table 5 was added to the solution, then the solution was adjusted to pH 2.0 with hydrochloric acid, and sonicated until completely dissolved. The solution was adjusted to the target pH as provided in table 5 with hydrochloric acid or sodium hydroxide. Finally, purified water was added to produce a final volume of 100 ml.

TABLE 5
Ingredient contents of sample 6 and sample 7

| | | |
|---|---|---|
| Ingredients | Sample 6 | Sample 7 |

| | | | | |
|---|---|---|---|---|
| Remdesivir | 500 | mg | 2,000 | mg |
| Sulfobutylether β-Cyclodextrin | 5,000 | mg | 10,000 | mg |
| (SBECD) | | | | |

| | | | |
|---|---|---|---|
| Sodium chloride | 300 | mg | 0 |

| | | |
|---|---|---|
| Hydrochloric acid or sodium | To pH 4.5 | To pH 3.5 |
| hydroxide | | |
| Purified water | Added to | Added to |
| | 100 ml | 100 ml |

### Example 4

Aerodynamic Particle Size Distribution of Nebulization Inhalation Solution (Sample 6 in Example 3):

Sample 6 was sprayed by a mesh based atomizer. The aerodynamic particle size distribution of droplets of sample 6 was measured on a Next Generation Impactor (NGI). The Next Generation Impactor was operated at a flow rate of 15 L/min for determination of the particle size distribution. For each of the impactor experiments, the impactor collection stages were coated with a silicone oil. The particle size distribution is expressed in terms of mass median aerodynamic diameter (MMAD) and Geometric Standard Deviation (GSD). The MMAD of remdesivir was less than 10 The GSD of remdesivir was less than 5% (Table 6).

TABLE 6
Aerodynamic particle size distribution of nebulization
inhalation solution (Sample 6 in Example 3)

| MMAD (μm) | GSD (%) |
|---|---|
| 3.64 | 1.55 |

### Example 5

Sample 6 in Example 3 was sprayed using a mesh based atomizer. A Malvern Spraytec (STP5313) was used to measure the particle size of the resulting droplets. The results are shown in Table 7.

TABLE 7
Droplet Particle size distribution of Sample
6 in Example 3 by using a mesh based atomizer

| Test time | Dv(50) μm |
|---|---|
| 1 | 5.9 |
| 2 | 4.9 |
| 3 | 4.3 |

### Example 6

The thermal stability of sample 6 in example 3 is shown in Table 8 below.

TABLE 8
Thermal stability of sample 6 in Example 3

| | 60° C. | | 40° C. | |
|---|---|---|---|---|
| | 0 days | 5 days | 0 days | 5 days |
| Degradation impurity | ND | 2.85% | ND | 0.08% |

ND: not detected

### Example 7

The thermal stability of sample 7 in example 3 is shown in Table 9 below.

TABLE 9
Thermal stability of sample 7 in Example 3

| | 0 days | 40° C. 5 days |
|---|---|---|
| Degradation impurity | ND | 0.25% |

ND: not detected

While various embodiments of the present invention have been described above, it should be understood that they have been presented by way of example only, and not limitation. For example, the present invention is not limited to the physical arrangements or dimensions illustrated or described. Nor is the present invention limited to any particular design or materials of construction. As such, the breadth and scope of the present invention should not be limited to any of the above-described exemplary embodiments, but should be defined only in accordance with the following claims and their equivalents.

What is claimed is:

1. A pharmaceutical formulation comprising:
(a) remdesivir or a pharmaceutically acceptable salt thereof;
(b) an excipient selected from the group consisting of (i) a pharmacologically acceptable stabilizer or complexing agent and (ii) a solubility enhancing agent; and
(c) a solvent,
wherein the pharmaceutical formulation has a pH of between about 2.0 and about 6.0.

2. The pharmaceutical formulation of claim 1, wherein the pharmaceutical formulation has a pH of between about 3.0 and about 5.0.

3. The pharmaceutical formulation of claim 1, wherein the pharmaceutical formulation has a pH of between about 3.5 and about 4.5.

4. The pharmaceutical formulation of claim 1, comprising a solubility enhancing agent selected from the group consisting of cyclodextrin derivatives, sulfobutylether β-cyclodextrin or one of the known pharmaceutically acceptable salts thereof, and combinations thereof.

5. The pharmaceutical formulation of claim 1, comprising a solubility enhancing agent selected from the group consisting of polysorbate 20, polysorbate 80, poloxamer, cyclodextrin derivatives, sodium dodecyl sulfate (SDS), sodium laurel sulfate, sodium octyl glycoside, polyethyl glycol, polypropyl glycol, copolymers, and combinations thereof.

6. The pharmaceutical formulation of to claim 4, wherein the solubility enhancing agent is sulfobutylether β-cyclodextrin or one of the known pharmaceutically acceptable salts thereof.

7. The pharmaceutical formulation of claim 6, wherein the concentration of sulfobutylether β-cyclodextrin is about 10 g/100 ml.

8. The pharmaceutical formulation of claim 1, wherein the formulation is suitable for administration by soft mist inhalation.

9. The pharmaceutical formulation of claim 1, wherein remdesivir or its pharmaceutically acceptable salt is present in a concentration between about 1 g/100 ml and about 20 g/100 ml.

10. The pharmaceutical formulation of claim 9, wherein remdesivir or its pharmaceutically acceptable salt is present in a concentration between about 10 g/100 ml and about 15 g/100 ml.

11. The pharmaceutical formulation of claim 1, wherein remdesivir is present in an amount between about 1 mg and about 100 mg.

12. The pharmaceutical formulation of claim 11, wherein remdesivir is present in an amount between about 10 mg and about 50 mg.

13. The pharmaceutical formulation of claim 10, wherein remdesivir is present in an amount between about 20 mg and about 30 mg.

14. The pharmaceutical formulation of claim 1, wherein the stabilizer or complexing agent is selected from the group consisting of edetic acid (EDTA) or one of the known salts thereof, disodium edetate, and edetate disodium dehydrate.

15. The pharmaceutical formulation of claim 14, wherein the stabilizer or complexing agent is present in a concentration between about 1 mg/100 ml and about 500 mg/100 ml.

16. The pharmaceutical formulation of claim 15, wherein the stabilizer or complexing agent is present in a concentration between about 5 mg/100 ml and about 200 mg/100 ml.

17. The pharmaceutical formulation of claim 15, wherein the stabilizer or complexing agent comprises edetate disodium dihydrate, which is present in a concentration of about 10 mg/100 ml.

18. The pharmaceutical formulation of claim 1 further comprising a preservative selected from the group consisting of benzalkonium chloride, benzoic acid, and sodium benzoate.

19. The pharmaceutical formulation of claim 18, wherein the preservative is benzalkonium chloride, which is present in a concentration between about 2 mg/100 ml and about 300 mg/100 ml.

20. The pharmaceutical formulation of claim 1, wherein the formulation is suitable for administration by nebulization.

21. The pharmaceutical formulation of claim 1, wherein remdesivir or its pharmaceutically acceptable salt is present in a concentration between about 100 mg/100 ml and about 10 g/100 ml.

22. The pharmaceutical formulation of claim 21, wherein remdesivir or its pharmaceutically acceptable salt is present in a concentration between about 1,000 mg/100 ml and about 5,000 mg/100 ml.

23. The pharmaceutical formulation of claim 1, wherein the solvent is water.

24. A method for administering the pharmaceutical formulation of claim 1, comprising nebulizing a defined amount of the pharmaceutical formulation using a device selected from a soft mist inhaler and a nebulization device.

25. The method according to claim 24, wherein the defined amount of the pharmaceutical formulation is less than about 70 microliters.

26. The method according to claim 25, wherein the defined amount of the pharmaceutical formulation is less than about 10 microliters.

27. A method of treating a viral infection in a patient comprising administering the pharmaceutical formulation of claim 1 to the patient, wherein the viral infection is selected from the group consisting of Ebola and Marburg virus (Filoviridae), coronavirus, SARS-CoV-2, Ross River virus, chikungunya virus, Sindbis virus, eastern equine encephalitis virus (Togaviridae, Alphavirus), vesicular stomatitis virus (Rhabdoviridae, Vesiculovirus), Amapari virus, Pichindé virus, Tacaribe virus, Junin virus, Machupo virus (Arenaviridae, Mammarenavirus), West Nile virus, dengue virus, yellow fever virus (Flaviviridae, Flavivirus), human immunodeficiency virus type 1 (Retroviridae, Lentivirus), Moloney murine leukemia virus (Retroviridae, Gammaretrovirus), influenza A virus (Orthomyxoviridae), respiratory syncytial virus (Paramyxoviridae, Pneumovirinae, Pneumovirus), vaccinia virus (Poxviridae, Chordopoxvirinae, Orthopoxvirus), herpes simplex virus type 1, herpes simplex virus type 2 (Herpesviridae, Alphaherpesvirinae, Simplexvirus), human cytomegalovirus (Herpesviridae, Betaherpesvirinae, Cytomegalovirus), Autographa californica nucleopolyhedrovirus (Baculoviridae, Alphabaculoviridae) (an insect virus), Semliki Forest virus, O'nyong-nyong virus, Sindbis virus, eastern/western/Venezuelan equine encephalitis virus (Togaviridae, Alphavirus), rubella (German measles) virus (Togaviridae, Rubivirus), rabies virus, Lagos bat virus, Mokola virus (Rhabdoviridae, Lyssavirus), Guanarito virus, Sabia virus, Lassa virus (Arenaviridae, Mammarenavirus), Zika virus, Japanese encephalitis virus, St. Louis encephalitis virus, tick-borne encephalitis virus, Omsk hemorrhagic fever virus, Kyasanur Forest virus (Flaviviridae, Flavivirus), human hepatitis C virus (Flaviviridae, Hepacivirus), influenza AB virus (Orthomyxoviridae, the common ‘flu’ virus), respiratory syncytial virus (Paramyxoviridae, Pneumovirinae, Pneumovirus), Hendra virus, Nipah virus (Paramyxoviridae, Paramyxovirinae, Henipavirus), measles virus (Paramyxoviridae, Paramyxovirinae, Morbillivirus), variola major (smallpox) virus (Poxviridae, Chordopoxvirinae, Orthopoxvirus), human hepatitis B virus (Hepadnaviridae, Orthohepadnavirus), hepatitis delta virus (hepatitis D virus), Middle East Respiratory Syndrome (MERS) virus, severe acute respiratory syndrome CoV (SARS-CoV).

28. The method of claim 27, wherein the pharmaceutical formulation is administered by soft mist or nebulization inhalation.
